(19) United States
(12) Patent Application Publication
(10) Pub. No.: US 2023/0086639 A1
(43) Pub. Date: Mar. 23, 2023

(54) Introducer Adapters, Introducer Assemblies, and Methods

(71) Applicant: Bard Access Systems, Inc., Salt Lake City, UT (US)

(72) Inventor: Glade H. Howell, Draper, UT (US)

(21) Appl. No.: 17/949,734

(22) Filed: Sep. 21, 2022

Related U.S. Application Data
(60) Provisional application No. 63/246,700, filed on Sep. 21, 2021.

(51) Int. Cl. A61M 25/00; A61M 25/06; A61M 39/06

(52) U.S. Cl. CPC A61M 25/0097; A61M 25/0606; A61M 39/06; A61M 2039/062

(57) ABSTRACT

Disclosed are introducer adapters, introducer assemblies, and methods. In an example, an introducer assembly can include a syringe, a needle, and an introducer adapter fluidly coupled therebetween. The introducer adapter can include a primary conduit, a secondary conduit extending from a side of the primary conduit, and a valve including an elastomeric septum sealing a proximal portion of the primary conduit or the secondary conduit. An access guidewire is optionally loaded in the introducer adapter and sealed therein by the valve. In another example, a method can include a method for securing vascular access with the foregoing introducer assembly. Such a method can include obtaining the introducer assembly, establishing a needle tract from an area of skin to a blood-vessel lumen of a patient with the needle, and advancing at least a guidewire tip of the access guidewire into the blood-vessel lumen for the securing of the vascular access.

### PRIORITY

This application claims the benefit of priority to U.S. Provisional Patent Application No. 63/246,700, filed Sep. 21, 2021, which is incorporated by reference in its entirety into this application.

### BACKGROUND

A guidewire is typically placed in a blood vessel with an introducer assembly before inserting a central venous catheter (“CVC”) or the like into the blood vessel over the guidewire. The introducer assembly typically includes a needle connected to a syringe. Upon accessing the blood vessel with the introducer assembly, the needle must be disconnected from the syringe to allow insertion of the guidewire through a needle hub of the needle and, subsequently, placement of the guidewire in the blood vessel. Both disconnecting the needle from the syringe and inserting the guidewire into the needle hub risk puncturing a backwall of the blood vessel, losing access to the blood vessel, or both due to overhandling the needle. What is needed is an adapter that does not require the needle to be disconnected from the syringe for placing the guidewire in the blood vessel.

Disclosed herein are introducer adapters, introducer assemblies, and methods that address the foregoing.

### SUMMARY

Disclosed herein is an introducer assembly including, in some embodiments, a syringe, a needle, and an introducer adapter fluidly coupling the syringe and the needle together. The introducer adapter includes a primary conduit, a secondary conduit extending from a side of the primary conduit, and a valve including an elastomeric septum sealing a proximal portion of the primary conduit or the secondary conduit. An access guidewire is optionally loaded in the introducer adapter and sealed therein by the valve.

In some embodiments, the valve is a non-elastomeric cylindrical or conical insert with the septum sealing an opening of the insert. The valve is disposed in the proximal portion of the primary conduit or the secondary conduit.

In some embodiments, the valve is an elastomeric grommet with the septum integral therewith sealing a cavity of the grommet. The valve is disposed in the proximal portion of the primary conduit or the secondary conduit.

In some embodiments, the septum is recessed in the proximal portion of the primary conduit or the secondary conduit.

In some embodiments, the valve seals the proximal portion of the secondary conduit.

In some embodiments, the valve seals the proximal portion of the primary conduit.

In some embodiments, an introducer-adapter bore in the proximal portion of the primary conduit includes a Luer taper complementing that of a syringe tip of the syringe.

In some embodiments, an introducer-adapter bore in the proximal portion of the secondary conduit includes a Luer taper complementing that of a syringe tip of the syringe.

In some embodiments, an introducer-adapter tip in a distal portion of the introducer adapter includes a Luer taper complementing that of a needle-hub bore of a needle hub of the needle.

In some embodiments, the introducer assembly further includes a guidewire-management device including the access guidewire. The guidewire-management device is optionally coupled to the introducer adapter.

In some embodiments, the guidewire-management device further includes a handle and a guidewire conduit. The handle includes a proximal sleeve, a distal sleeve, and a gap between the proximal and distal sleeves configured to provide access to the access guidewire for handling the access guidewire. The guidewire conduit is coupled to the proximal sleeve. The guidewire conduit is configured to provide storage of the access guidewire in a sterile state thereof.

In some embodiments, a distal portion of the distal sleeve includes an integral septum-piercing tip. The septum-piercing tip is configured to pierce the septum of the valve and deliver the access guidewire therethrough.

In some embodiments, the guidewire-management device further includes a septum-piercing tip coupled to a distal portion of the distal sleeve. The septum-piercing tip is configured to pierce the septum of the valve and deliver the access guidewire therethrough.

In some embodiments, the access guidewire includes a bare-wire portion around which the septum seals.

In some embodiments, the access guidewire includes a wound-wire portion terminating with an atraumatic ‘J’-shaped guidewire tip.

Also disclosed herein is an introducer adapter including, in some embodiments, a primary conduit, a secondary conduit extending from a side of the primary conduit, and a valve including an elastomeric member for sealing a proximal portion of the primary conduit or the secondary conduit.

In some embodiments, the valve is a non-elastomeric cylindrical or conical insert with an elastomeric septum for sealing an opening of the insert. The valve is disposed in the proximal portion of the primary conduit or the secondary conduit.

In some embodiments, the valve is an elastomeric grommet with an elastomeric septum integral therewith for sealing a cavity of the grommet. The valve is disposed in the proximal portion of the primary conduit or the secondary conduit.

In some embodiments, the septum is recessed in the proximal portion of the primary conduit or the secondary conduit.

In some embodiments, the valve is an integrated Touhy-Borst valve including an elastomeric ‘O’-ring for sealing the valve. The valve is integrated into the proximal portion of the primary conduit or the secondary conduit.

In some embodiments, the valve is a Touhy-Borst adapter including an elastomeric ‘O’-ring for sealing the valve. The adapter is coupled to the proximal portion of the primary conduit or the secondary conduit.

In some embodiments, the valve is for sealing the proximal portion of the secondary conduit.

In some embodiments, the valve is for sealing the proximal portion of the primary conduit.

In some embodiments, an introducer-adapter bore in the proximal portion of the primary conduit includes a Luer taper complementing that of a syringe tip of the syringe.

In some embodiments, an introducer-adapter bore in the proximal portion of the secondary conduit includes a Luer taper complementing that of a syringe tip of the syringe.

In some embodiments, an introducer-adapter tip in a distal portion of the introducer adapter includes a Luer taper complementing that of a needle-hub bore of a needle hub of the needle.

Also disclosed herein is a method for securing vascular access. The method includes, in some embodiments, an introducer assembly-obtaining step, a needle tract-establishing step, and an access guidewire-advancing step. The introducer assembly-obtaining step includes obtaining an introducer assembly. The introducer assembly includes a syringe, a needle, and an introducer adapter fluidly coupling the syringe and the needle together. The introducer adapter includes a primary conduit, a secondary conduit extending from a side of the primary conduit, and a valve including an elastomeric septum sealing a proximal portion of the primary conduit or the secondary conduit. The needle tract-establishing step includes establishing a needle tract from an area of skin to a blood-vessel lumen of a patient with the needle. needle tract-establishing step. The access guidewire-advancing step includes advancing at least a guidewire tip of an access guidewire into the blood-vessel lumen for the securing of the vascular access.

In some embodiments, the method further includes an introducer assembly-adjusting step. The introducer assembly-adjusting step includes adjusting the introducer assembly such that the introducer assembly is in a ready-to-deploy state thereof. In the ready-to-deploy state of the introducer assembly, the guidewire tip of the access guidewire is just proximal of a needle tip in a distal portion of a needle shaft of the needle. This allows for the advancing of the access guidewire into the blood-vessel lumen immediately upon the establishing of the needle tract.

In some embodiments, the method further includes a septum-piercing step. The septum-piercing step includes piercing the septum with an integral septum-piercing tip of a distal portion of a distal sleeve of a guidewire-management device. The septum-piercing step is performed before the introducer assembly-adjusting step.

In some embodiments, the method further includes a septum-piercing step. The septum-piercing step includes piercing the septum with a septum-piercing tip coupled to a distal portion of a distal sleeve of a guidewire-management device. The septum-piercing step is performed before the introducer assembly-adjusting step.

In some embodiments, the access guidewire-advancing step allows the guidewire tip to transition from a straightened state in the needle to a curved state in the blood-vessel lumen.

In some embodiments, the method further includes a guidewire-management-device-withdrawing step. The guidewire-management-device-withdrawing step includes withdrawing the guidewire-management device from the introducer adapter after the access guidewire-advancing step. The guidewire-management-device-withdrawing step leaves the septum to seal around a bare-wire portion of the access guidewire.

In some embodiments, the method further includes a blood-aspirating step. The blood-aspirating step includes aspirating blood with the syringe to confirm the establishing of the needle tract. The seal of the septum around the bare-wire portion of the access guidewire is sufficient to maintain a vacuum during the blood-aspirating step.

In some embodiments, the method further includes a needle-withdrawing step. The needle-withdrawing step includes withdrawing the needle from the patient but leaving the access guidewire in the blood-vessel lumen. The needle-withdrawing step includes withdrawing both the introducer adapter and the needle over a proximal portion of the access guidewire until a proximal end of the access guidewire escapes from the needle.

In some embodiments, the needle-withdrawing step includes holding the access guidewire in place at or near the area of skin including the needle tract while withdrawing both the introducer adapter and the needle over the proximal portion of the access guidewire.

In some embodiments, the method further includes a syringe-disconnecting step. The syringe-disconnecting step includes disconnecting the syringe before the needle-withdrawing step, thereby preventing creation of a vacuum during the needle-withdrawing step.

These and other features of the concepts provided herein will become more apparent to those of skill in the art in view of the accompanying drawings and following description, which describe particular embodiments of such concepts in greater detail.

### DRAWINGS

FIG. 1 illustrates an introducer assembly including an introducer adapter coupling together a needle, a syringe, and a guidewire-management device in a ready-to-deploy state of the introducer assembly in accordance with some embodiments.

FIG. 2 illustrates the introducer assembly of FIG. 1 with the guidewire-management device partially withdrawn from the introducer adapter in accordance with some embodiments.

FIG. 3 illustrates a detailed view of a longitudinal cross section of the introducer assembly of FIG. 1 in accordance with some embodiments.

FIG. 4 illustrates a detailed view of a longitudinal cross section of the introducer assembly of FIG. 2 in accordance with some embodiments.

FIG. 5 illustrates another detailed view of the longitudinal cross section of the introducer assembly of FIG. 2 in accordance with some embodiments.

FIG. 6 illustrates a detailed view of a longitudinal cross section of the introducer assembly in which an integral septum-piercing tip of the guidewire-management device passes through a septum of an insert of the introducer adapter in accordance with some embodiments.

FIG. 7 illustrates a detailed view of a longitudinal cross section of the introducer assembly in which a septum-piercing tip coupled to the guidewire-management device is poised to pass through the septum of the insert of the introducer adapter in accordance with some embodiments.

FIG. 8 illustrates a detailed view of a longitudinal cross section of the introducer adapter in accordance with some embodiments.

FIG. 9A illustrates a first valve in accordance with some embodiments.

FIG. 9B illustrates a second valve in accordance with some embodiments.

FIG. 9C illustrates a third valve in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a catheter includes a portion or section of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal section, or the proximal length of the catheter can include the proximal end of the catheter; however, the proximal portion, the proximal section, or the proximal length of the catheter need not include the proximal end of the catheter. That is, unless context suggests otherwise, the proximal portion, the proximal section, or the proximal length of the catheter is not a terminal portion or terminal length of the catheter.

With respect to “distal,” a “distal portion” or a “distal section” of, for example, a catheter includes a portion or section of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal section, or the distal length of the catheter can include the distal end of the catheter; however, the distal portion, the distal section, or the distal length of the catheter need not include the distal end of the catheter. That is, unless context suggests otherwise, the distal portion, the distal section, or the distal length of the catheter is not a terminal portion or terminal length of the catheter.

Unless defined otherwise, all technical and scientific terms used herein have the same meaning as commonly understood by those of ordinary skill in the art.

As set forth above, a guidewire is typically placed in a blood vessel with an introducer assembly before inserting a CVC or the like into the blood vessel over the guidewire. The introducer assembly typically includes a needle connected to a syringe. Upon accessing the blood vessel with the introducer assembly, the needle must be disconnected from the syringe to allow insertion of the guidewire through a needle hub of the needle and, subsequently, placement of the guidewire in the blood vessel. Both disconnecting the needle from the syringe and inserting the guidewire into the needle hub risk puncturing a backwall of the blood vessel, losing access to the blood vessel, or both due to overhandling the needle. What is needed is an adapter that does not require the needle to be disconnected from the syringe for placing the guidewire in the blood vessel.

Disclosed herein are introducer adapters, introducer assemblies, and methods that do not require disconnecting a needle from a syringe for placing a guidewire in a blood vessel like typical introducer assemblies. Such introducer adapters, introducer assemblies, and methods are advantageous in that they do not have the same risk of puncturing a backwall of a blood vessel or losing access to the blood vessel due to overhandling. In an example, an introducer assembly can include a syringe, a needle, and an introducer adapter fluidly coupling the syringe and the needle together. The introducer adapter can include a primary conduit, a secondary conduit extending from a side of the primary conduit, and a valve including an elastomeric septum sealing a proximal portion of the primary conduit or the secondary conduit. An access guidewire is optionally loaded in the introducer adapter and sealed therein by the valve. In another example, a method can include a method for securing vascular access with the foregoing introducer assembly. Such a method can include an introducer assembly-obtaining step of obtaining the introducer assembly, a needle tract-establishing step of establishing a needle tract from an area of skin to a blood-vessel lumen of a patient with the needle, and an access guidewire-advancing step of advancing at least a guidewire tip of the access guidewire into the blood-vessel lumen for the securing of the vascular access. Again, these and other features will become more apparent in view of the accompanying drawings and following description, which describe particular embodiments in greater detail.

### Introducer Assemblies

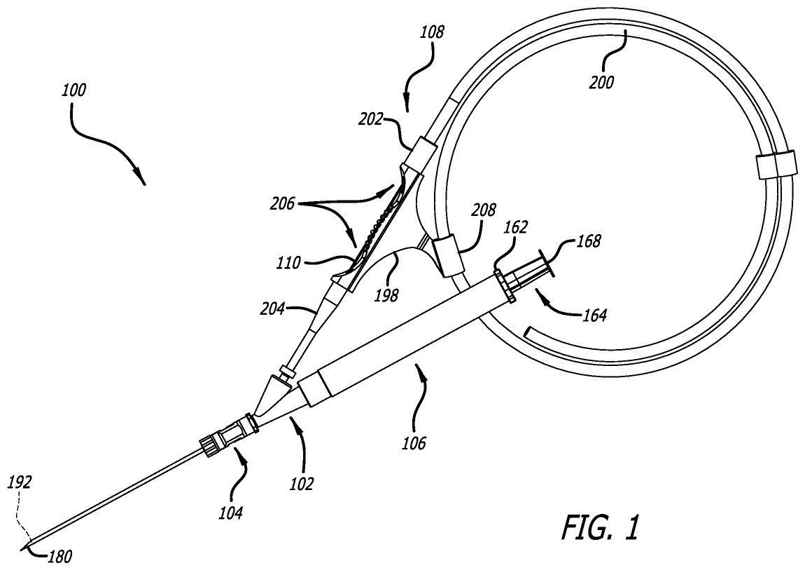

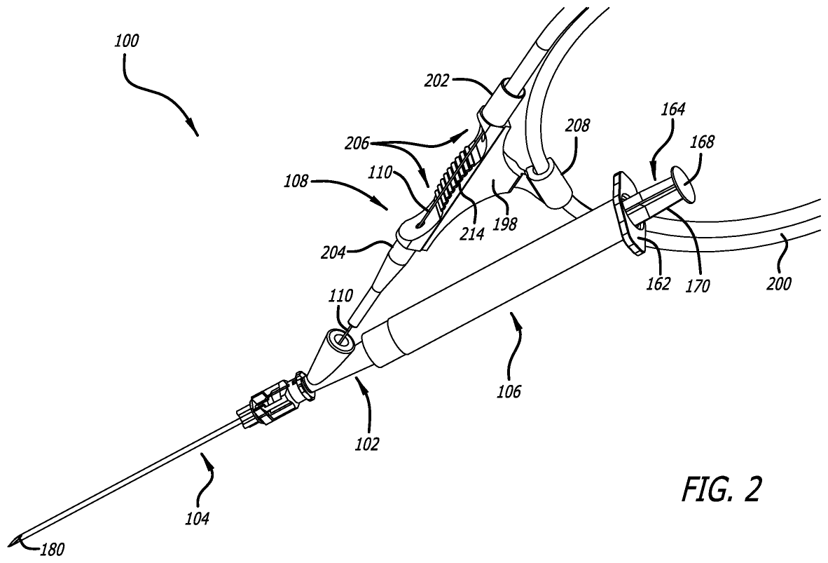

FIGS. 1 and 2 illustrate different views of an introducer assembly 100 in accordance with some embodiments. Indeed, FIG. 1 illustrates the introducer assembly 100 including an introducer adapter 102 coupling together a needle 104, a syringe 106, and a guidewire-management device 108 including an access guidewire 110 in a ready-to-deploy state of the introducer assembly 100. FIG. 2 illustrates the introducer assembly 100 with the guidewire-management device 108 partially withdrawn from the introducer adapter 102.

The introducer assembly 100 includes at least the syringe 106 (or another vacuum-generating device), the needle 104, and the introducer adapter 102 fluidly coupling the syringe 106 (or the other vacuum-generating device) and the needle 104 together in the ready-to-deploy state of the introducer assembly 100. Neither the access guidewire 110 nor the guidewire-management device 108, which, in some embodiments, provides the access guidewire 110, needs to be present in the ready-to-deploy state of the introducer assembly 100. This is because the access guidewire 110 can be inserted into the introducer assembly 100, optionally, by way of the guidewire-management device 108, and subsequently advanced into a blood-vessel lumen of a patient upon establishing a needle tract thereto with the needle 104. However, the ready-to-deploy state of the introducer assembly 100 including the access guidewire 110 is advantageous when the access guidewire 110 is loaded in a combination of the introducer adapter 102 and the needle 104 and sealed therein by the valve 116 of the introducer adapter 102. Indeed, as shown, the access guidewire 110 can be loaded in the needle lumen of the needle 104 such that the guidewire tip 192 of the access guidewire 110 is located just proximal of the needle tip 180 of the needle 104. In this way, the access guidewire 110 is available to be immediately and directly advanced into the blood-vessel lumen of the patient upon establishing the needle tract without having to further handle the introducer assembly 100 to insert the access guidewire 110.

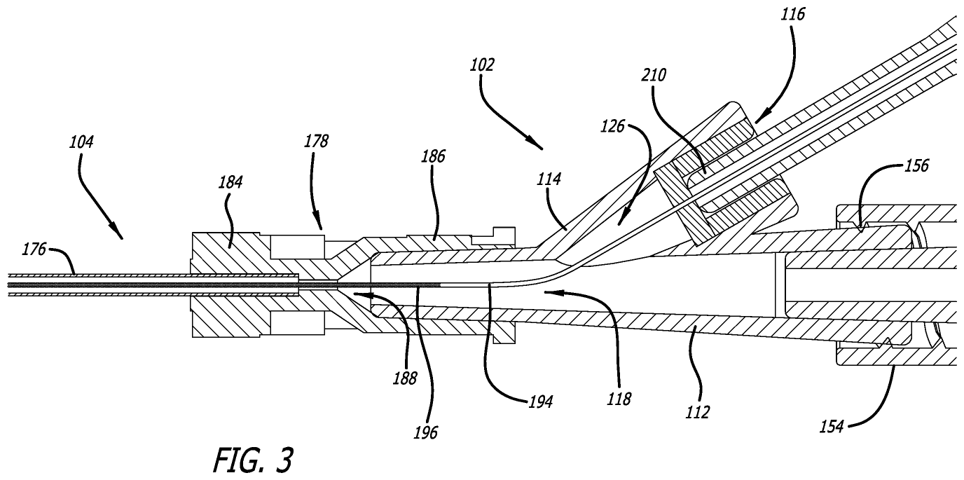

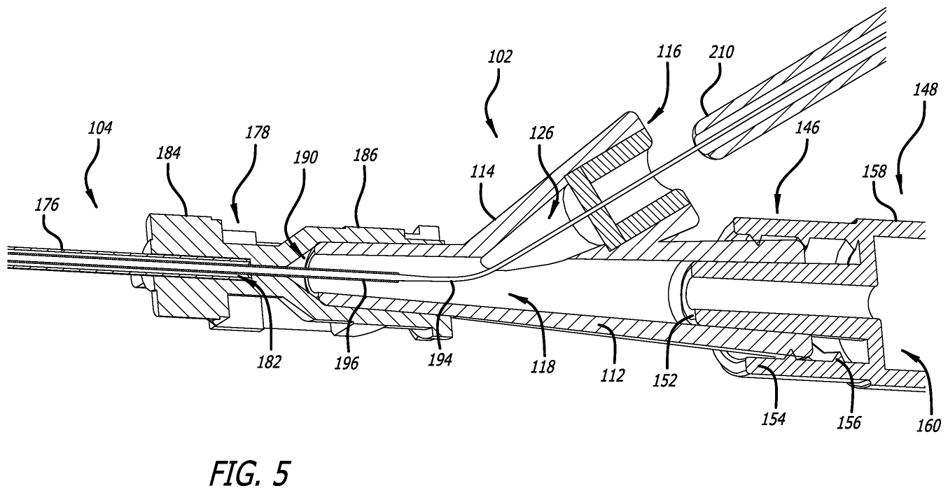

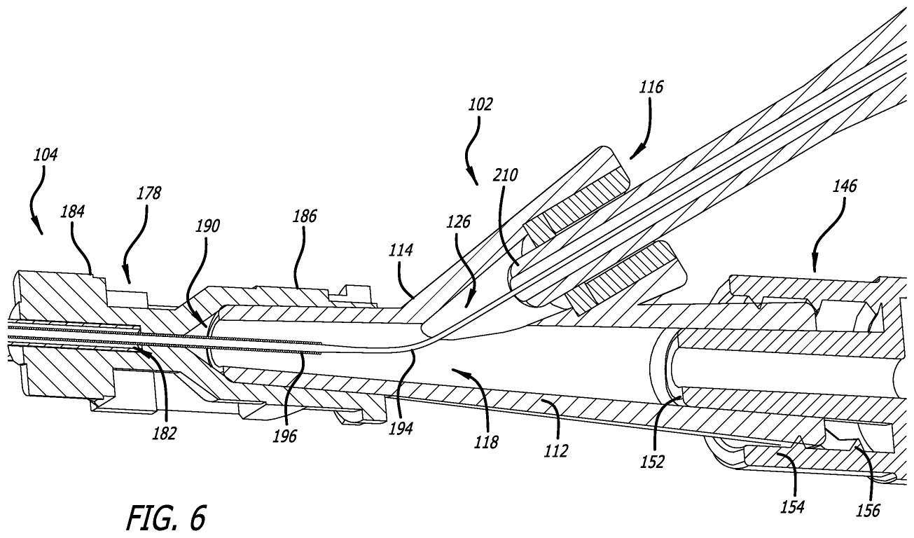

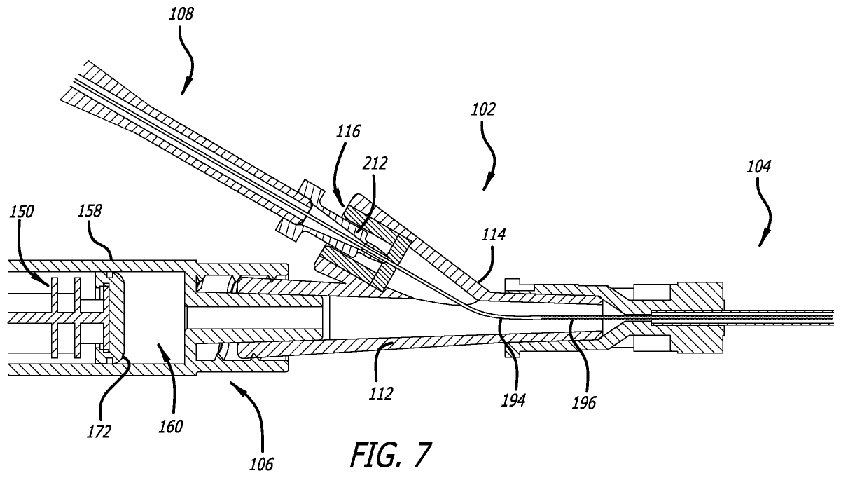

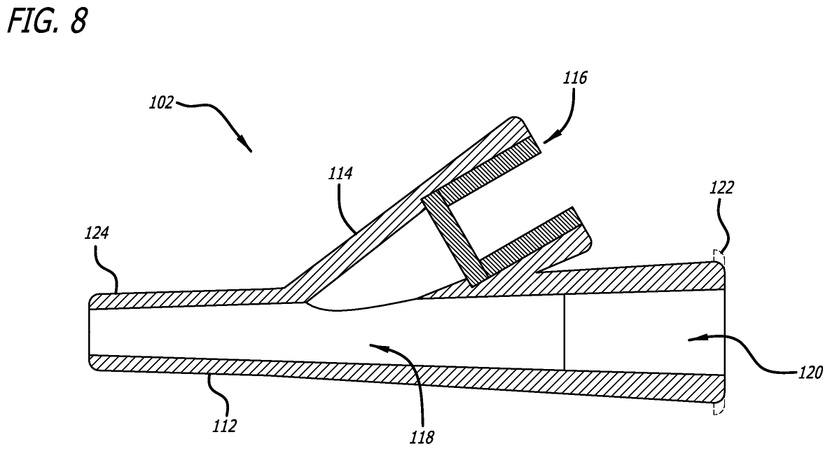

FIGS. 3-8 illustrate different views of the introducer assembly 100 and the introducer adapter 102 thereof by way of different longitudinal cross sections.

As shown, the introducer adapter 102 includes a primary conduit 112, a secondary conduit 114 extending from a side of the primary conduit 112 at an angle (e.g., 30° angle formed between axes of the primary and secondary conduits 112 and 114), and a valve 116 including an elastomeric member as set forth below for sealing a proximal portion of the primary conduit 112 or the secondary conduit 114.

Notably, the introducer adapter 102 can have a primary configuration for fluidly coupling the syringe 106 to the primary conduit 112, as shown, or a secondary configuration for fluidly coupling the syringe 106 to the secondary conduit 114. Correspondingly, the primary configuration of the introducer adapter 102 is for inserting the access guidewire 110 into the secondary conduit 114, as shown, while the secondary configuration of the introducer adapter 102 is for inserting the access guidewire 110 into the primary conduit 112—the inserting of the access guidewire 110 into the secondary conduit 114 or the primary conduit 112, optionally, by way of the guidewire-management device 108. When the syringe 106 is fluidly coupled to the primary conduit 112 and the access guidewire 110 is inserted into the secondary conduit 114 in accordance with the primary configuration of the introducer adapter 102, the syringe 106 is axially aligned with the needle 104 through the primary conduit 112, thereby ensuring a timely and pronounced blood flashback into at least the syringe tip 152 upon establishing a needle tract to a blood-vessel lumen with the needle 104. When the syringe 106 is fluidly coupled to the secondary conduit 114 and the access guidewire 110 is inserted into the primary conduit 112 in accordance with the secondary configuration of the introducer adapter 102, the syringe 106 is not axially aligned with the needle 104 through the primary conduit 112, thereby possibly compromising such a timely and pronounced blood flashback. That said, in the secondary configuration of the introducer adapter 102, the access guidewire 110 can be advantageously advanced straight into the blood-vessel lumen through the primary conduit 112 without bending, thereby facilitating the advancing of the access guidewire 110 into the blood-vessel lumen.

The primary conduit 112 includes a primary channel 118 extending through both proximal and distal ends of the primary conduit 112. In the primary configuration of the introducer adapter 102, a proximal portion of the primary conduit 112 or the primary channel 118 thereof includes an introducer-adapter bore 120 having a Luer taper (e.g., a 6% taper) complementing that of the syringe tip 152 of the syringe 106. Optionally, the proximal portion of the primary conduit 112 also includes a primary introducer-adapter flange 122 configured to screw together with the internal threads 156 of the threaded collar 154 of the syringe hub 146. However, in the secondary configuration of the introducer adapter 102, the proximal portion of the primary conduit 112 or the primary channel 118 thereof instead includes the valve 116 disposed therein. Alternatively, the proximal portion of the primary conduit 112 instead includes the valve 116 coupled thereto. For example, the valve 116 can be the Touhy-Borst adapter set forth below with the internal threads of the adapter hub 142 screwed together with the primary introducer-adapter flange 122. Whether the primary or secondary configuration of the introducer adapter 102, a distal portion of the introducer adapter 102 or the primary conduit 112 thereof includes an introducer-adapter tip 124 having a Luer taper (e.g., a 6% taper) complementing that of the needle-hub bore 190 of the needle hub 178 of the needle 104. While not shown, the introducer-adapter tip 124 optionally includes a threaded collar with internal threads like that of the syringe 106 set forth below, the threaded collar around the introducer-adapter tip 124 configured for forming a so-called Luer lock-style connection with the optional needle-hub flange of the needle hub 178 of the needle 104 as set forth below.

The secondary conduit 114 includes a secondary channel 126 extending through a proximal end of the secondary conduit 114 and into a medial portion of the primary channel 118 of the primary conduit 112. In the primary configuration of the introducer adapter 102, a proximal portion of the secondary conduit 114 or the secondary channel 126 thereof includes the valve 116 disposed therein. Alternatively, the proximal portion of the secondary conduit 114 includes the valve 116 coupled thereto. For example, the valve 116 can be the Touhy-Borst adapter set forth below with the internal threads of the adapter hub 142 screwed together with a secondary introducer-adapter flange (not shown) akin to the primary introducer-adapter flange 122. However, in the secondary configuration of the introducer adapter 102, the proximal portion of the secondary conduit 114 or the secondary channel 126 thereof instead includes the introducer-adapter bore 120 having the Luer taper (e.g., a 6% taper) complementing that of the syringe tip 152 of the syringe 106. Optionally, the proximal portion of the secondary conduit 114 also includes the secondary introducer-adapter flange, which is also configured to screw together with the internal threads 156 of the threaded collar 154 of the syringe hub 146.

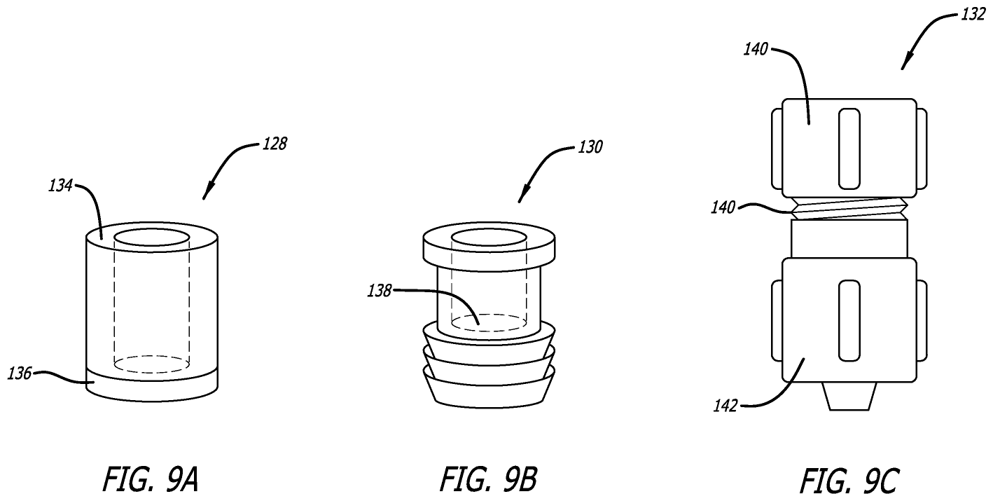

FIGS. 9A-9C illustrate valves 128, 130, and 132 for sealing the proximal portion of the primary conduit 112 or the secondary conduit 114 in accordance with some embodiments. It should be understood the valves 128, 130, and 132 are species of the valve 116 set forth above.

As shown in FIG. 9A, the valve 128 is a cylindrical or conical insert 134 with a septum 136 disposed therein or thereover for sealing an opening of the insert 134. The insert 134 can be formed of a relatively rigid, non-elastomeric material such as polyethylene, polypropylene, poly(methyl methacrylate), polyvinyl chloride, acrylonitrile butadiene styrene, or polycarbonate, whereas the septum 136 can be formed of an elastomeric material such as a silicone rubber, a natural rubber, a synthetic rubber, or a combination thereof such as a red rubber, which is a blend of natural rubber and styrene butadiene rubber. While the septum 136 is shown as intact and, therefore, configured to be pierced by the septum-piercing tip 210 or 212 of the guidewire-management device 108, the septum 136 can alternatively include a slit, a plurality of intersecting slits, or a through hole having a smaller inner diameter than an outer diameter of the access guidewire 110, through which a distal portion of the distal sleeve 204 of the guidewire-management device 108 can be used to deliver the access guidewire 110. The valve 128 is disposed in the proximal portion of the secondary conduit 114 in the primary configuration of the introducer adapter 102 or the proximal portion of the primary conduit 112 in the secondary configuration of the introducer adapter 102, notably with the septum 136 recessed in the proximal portion of the secondary conduit 114 or the primary conduit 112.

As shown in FIG. 9B, the valve 130 is a grommet with an integral septum 138 sealing an opening of the grommet, thereby forming a sealed cavity of the grommet. The grommet and the septum 138 thereof can be formed of an elastomeric material such as a silicone rubber, a natural rubber, a synthetic rubber, or a combination thereof such as a red rubber. Like that set forth for the septum 136, the septum 138 is shown as intact and, therefore, configured to be pierced by the septum-piercing tip 210 or 212 of the guidewire-management device 108; however, the septum 138 can alternatively include a slit, a plurality of intersecting slits, or a through hole having a smaller inner diameter than the outer diameter of the access guidewire 110, through which a distal portion of the distal sleeve 204 of the guidewire-management device 108 can be used to deliver the access guidewire 110. The valve 130 is disposed in the proximal portion of the secondary conduit 114 in the primary configuration of the introducer adapter 102 or the proximal portion of the primary conduit 112 in the secondary configuration of the introducer adapter 102, notably with the septum 138 recessed in the proximal portion of the secondary conduit 114 or the primary conduit 112.

As shown in FIG. 9C, the valve 132 is a Touhy-Borst adapter with an elastomeric ‘O’-ring or the like for sealing the Touhy-Borst adapter when compressed between an adapter nut 140 and an adapter hub 142 screwed together by way of their respective internal threads (not shown) and external threads 144. While not shown, the adapter hub 142 includes internal threads like the syringe hub 146 configured to screw together the proximal portion of the secondary conduit 114 in the primary configuration of the introducer adapter 102 or the proximal portion of the primary conduit 112 in the secondary configuration of the introducer adapter 102.

While not shown, the valve 132 can alternatively be integrated into the proximal portion of the secondary conduit 114 or the proximal portion of the primary conduit 112. Indeed, the valve 132 can alternatively be an integrated Touhy-Borst valve with the elastomeric ‘O’-ring or the like for sealing the Touhy-Borst valve when compressed between a valve nut (see, for example, the adapter nut 140) and the proximal portion of the secondary conduit 114 or the proximal portion of the primary conduit 112 (see, for example, the adapter hub 142) screwed together by way of internal threads of the valve nut and external threads of the proximal portion of the secondary conduit 114 or the proximal portion of the primary conduit 112. Such a valve is integrated into the proximal portion of the secondary conduit 114 in the primary configuration of the introducer adapter 102 or the proximal portion of the primary conduit 112 in the secondary configuration of the introducer adapter 102.

Notably, the access guidewire 110 can be used directly with the valve 132 without the guidewire-management device 108 because the seal of the valve 132 is the ‘O’-ring or the like compressed between the adapter nut 140 and the adapter hub 142 when screwed together—not the septum 136 or 138, for which the septum-piercing tip 210 or 212 or the like of the guidewire-management device 108 is configured to deliver the access guidewire 110 therethrough.

FIGS. 3-7 illustrate different views of the introducer assembly 100 and the syringe 106 thereof by way of different longitudinal cross sections; however, reference is also made to FIGS. 1 and 2, which also illustrate different views of the introducer assembly 100 and the syringe 106 thereof.

As shown, the syringe 106 includes a syringe hub 146, a barrel 148, and a plunger 150 disposed in the barrel 148 in at least the ready-to-deploy state of the introducer assembly 100.

The syringe hub 146 includes a syringe tip 152 extending from a distal portion (e.g., a distal end) of the barrel 148. In addition, the syringe hub 146 can include a threaded collar 154 extending from the distal portion (e.g., the distal end) of the barrel 148 around the syringe tip 152.

The syringe tip 152 is configured to insert into the introducer-adapter bore 120 in the proximal portion of the primary conduit 112 or the secondary conduit 114 for fluidly connecting the syringe 106 to the introducer adapter 102. Indeed, the syringe tip 152 can have a Luer taper (e.g., a 6% taper) configured to insert into the introducer-adapter bore 120, which introducer-adapter bore 120 is complementarily configured as set forth above.

The threaded collar 154 includes internal threads 156 configured to screw together with the primary introducer-adapter flange 122 or the secondary primary introducer-adapter flange (not shown) of the introducer adapter 102, each of which is optional as set forth above. When present, the threaded collar 154 of the syringe hub 146 advantageously provides a so-called Luer lock-style connection with the primary introducer-adapter flange 122 or the secondary primary introducer-adapter flange of the introducer adapter 102 for added security against inadvertent disconnection over that provided by an otherwise Luer slip-style connection.

The barrel 148 includes a barrel wall 158, a barrel chamber 160 defined by the barrel wall 158, and a barrel flange 162, barrel collar, or the like outwardly extending from a proximal portion (e.g., a proximal end) of the barrel 148 or barrel wall 158 configured for actuating the syringe 106 together with the plunger flange 168, the plunger collar, or the like set forth below.

The barrel chamber 160 is configured to accept the plunger 150 when inserted therein. Indeed, the barrel chamber 160 extends from a distal end of the barrel 148, which is a closed end of the barrel 148 (excepting the syringe tip 152), to the proximal end of the barrel 148, which is an open end of the barrel 148 into which the plunger 150 can be inserted.

The plunger 150 includes a one-piece plunger shaft 164, a piston 166 fitted over a distal portion (e.g., a distal end) of the plunger shaft 164, and a plunger flange 168, a plunger collar, or the like outwardly extending from a proximal portion (e.g., a proximal end) of the plunger 150 configured for actuating the syringe 106 together with the barrel flange 162, barrel collar, or the like.

The plunger shaft 164 can include orthogonal struts 170 meeting along their longitudinal edges at a central axis of the plunger shaft 164. However, the plunger shaft 164 can take other forms, so the plunger shaft 164 is not limited to the orthogonal struts 170.

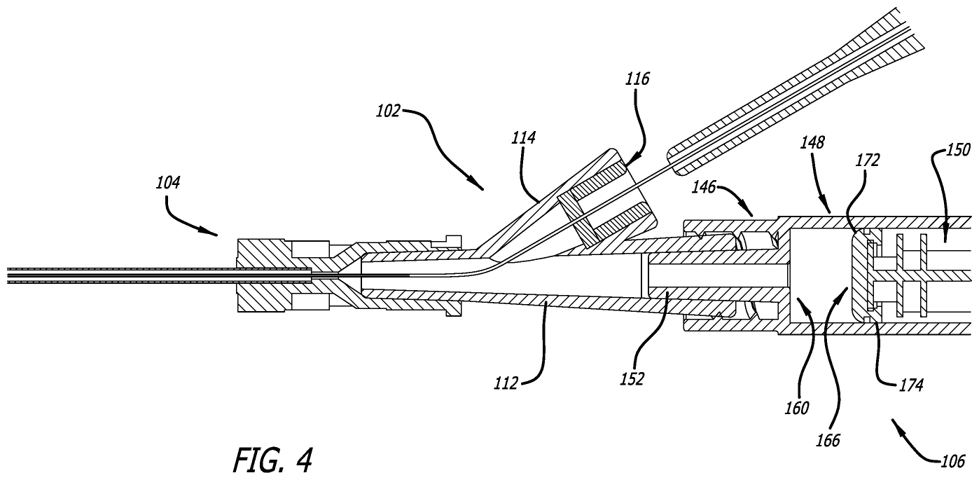

The piston 166, which can be an integral, elastomeric piston, includes one or more rings configured to respectively form one or more seals with the barrel wall 158. The one-or-more rings include at least a leading ring 172 configured to form a seal with the barrel wall 158. The one-or-more rings can also include a trailing ring 174 as shown in FIG. 4. Like the leading ring 172, the trailing ring 174 is configured to form a seal with the barrel wall 158. Indeed, the trailing ring 174, when present, provides a backup seal with the barrel wall 158. Together, the leading ring 172 and the trailing ring 174 ensure the seal (e.g., the seal provided by the leading ring 172, the trailing ring 174, or both the leading ring 172 and the trailing ring 174) between the piston 166 and the barrel wall 158 remains intact while the syringe 106 is actuated, thereby allowing the syringe 106 to consistently aspirate a liquid such as blood when the plunger 150 is withdrawn from the barrel 148.

FIGS. 3-7 illustrate different views of the introducer assembly 100 and the needle 104 thereof by way of different longitudinal cross sections; however, reference is also made to FIGS. 1 and 2, which also illustrate different views of the introducer assembly 100 and the needle 104 thereof.

As shown, the needle 104 includes a needle shaft 176 and a needle hub 178 over a proximal portion of the needle shaft 176 including a proximal end of the needle shaft 176.

The needle shaft 176 includes a needle tip 180 in a distal portion of the needle shaft 176 and a needle-shaft lumen 182 extending through an entirety of the needle shaft 176. Notably, the needle-shaft lumen 182 and the needle-hub lumen 188 set forth below together form a needle lumen.

The needle hub 178 includes a neck 184 and a needle-hub connector 186 in a proximal portion of the needle hub 178.

The neck 184 includes a neck portion of a needle-hub lumen 188 extending through an entirety of the needle hub 178. A remainder of the needle-hub lumen 188 is formed by the needle-hub bore 190, particularly that not occupied by the syringe tip 152 in the ready-to-deploy state of the introducer assembly 100. Notably, the needle-hub lumen 188 and the needle-shaft lumen 182 set forth above together form the needle lumen.

The needle-hub connector 186 includes a needle-hub bore 190, which is configured to accept the syringe tip 152 therein for fluidly connecting the needle 104 to the introducer adapter 102. Indeed, the needle-hub bore 190 can have a Luer taper (e.g., a 6% taper) configured to accept the introducer-adapter tip 124 therein, which introducer-adapter tip 124 is complementarily configured as set forth above.

While not shown, the needle hub 178 can include a needle-hub flange like the primary introducer-adapter flange 122 configured to screw together with the internal threads of the optional threaded collar around the introducer-adapter tip 124. When present, the needle-hub flange advantageously provides a so-called Luer lock-style connection with the internal threads of the threaded collar of the introducer-adapter tip 124 for added security against inadvertent disconnection over that provided by an otherwise Luer slip-style connection.

FIGS. 3-7 illustrate different views of the introducer assembly 100 and the access guidewire 110 thereof by way of different longitudinal cross sections; however, reference is also made to FIGS. 1 and 2, which also illustrate different views of the introducer assembly 100 and the syringe 106 thereof.

Again, the introducer assembly 100 can include the access guidewire 110 loaded therein in the ready-to-deploy state of the introducer assembly 100. When loaded in the introducer assembly 100 in the ready-to-deploy state thereof, the access guidewire 110 is loaded in the combination of the introducer adapter 102 and the needle 104 and sealed therein by the valve 116 of the introducer adapter 102. Indeed, as shown, the access guidewire 110 can be loaded in the needle lumen of the needle 104 such that the guidewire tip 192 of the access guidewire 110 is located just proximal of the needle tip 180 of the needle 104. In this way, the access guidewire 110 is available to be immediately and directly advanced into the blood-vessel lumen of the patient upon establishing the needle tract without having to further handle the introducer assembly 100 to insert the access guidewire 110.

The access guidewire 110 can include a guidewire tip 192 in the form of an atraumatic ‘J’-shaped guidewire tip configured to prevent puncturing a back wall of a blood vessel. Such a guidewire tip is configured to assume a straightened state in the needle 104 or the needle lumen thereof and a curved state when the guidewire tip 192 is advanced beyond the needle tip 180 of the needle 104 in a deployed state of the introducer assembly 100 such as when the guidewire tip 192 is advanced into a blood-vessel lumen.

The access guidewire 110 can further include a bare-wire portion 194 and a wound-wire portion 196 distal of the bare-wire portion 194, proximal of the bare-wire portion 194, or both. When the wound-wire portion 196 of the access guidewire 110 is distal of the bare-wire portion 194, the wound-wire portion 196 terminates with the guidewire tip 192. The bare-wire portion 194 of the access guidewire 110 distally extends through the valve 116 of the introducer adapter 102 in a number of states of the introducer assembly 100 such that the septum 136 or 138 or ‘O’-ring forms a fluid-tight seal around the bare-wire portion 194 of the access guidewire 110. For example, the bare-wire portion 194 of the access guidewire 110 distally extend through the valve 116 of the introducer adapter 102 to maintain the fluid-tight seal even when the guidewire tip 192 is advanced into a blood-vessel lumen in a deployed state of the introducer assembly 100. Notably, the access guidewire 110 need not have the bare-wire portion 194 and the wound-wire portion 196. At least the foregoing bare-wire portion 194 of the access guidewire 110 can instead be a flat-wound or ground-wound portion of the access guidewire 110, around which the septum 136 or 138 or ‘O’-ring can also form a fluid-tight seal. The flat-wound portion of the access guidewire 110, when present, includes windings of a tape instead of a round wire. The ground-wound portion of the access guidewire 110, when present, includes windings of a round wire ground down to flatten the windings.

Notably, if the access guidewire 110 is not provided by the guidewire-management device 108, the access guidewire 110 includes a proximal portion proximally extending from the valve 116 in at least the ready-to-deploy state of the introducer assembly 100. While not shown, the proximal portion of the access guidewire 110 can be disposed in a sterile barrier (e.g., a sterile bag) configured to maintain sterility of the access guidewire 110.

FIGS. 1 and 2 illustrate different views of the introducer assembly 100 and the guidewire-management device 108 thereof.

As shown, the guidewire-management device 108 includes a handle 198, a guidewire conduit 200, and the access guidewire 110 distributed between the handle 198 and the guidewire conduit 200.

The handle 198 includes a proximal sleeve 202, a distal sleeve 204, and a gap 206 between the proximal and distal sleeves 202 and 204.

The proximal sleeve 202 of the handle 198 includes a bore in a proximal portion of the proximal sleeve 202, which bore includes a distal end of the guidewire conduit 200 inserted therein, thereby coupling the guidewire conduit 200 to the handle 198. Notably, the handle 198 further includes a guidewire-conduit clip 208 opposite the gap 206 further coupling the guidewire conduit 200 to the handle 198.

The distal sleeve 204 of the handle 198 can include an integral septum-piercing tip 210 or a separate septum-piercing tip 212 coupled to a distal portion of the distal sleeve 204 configured to pierce the septum 136 or 138 of the valve 128 or 130) and deliver the access guidewire 110 therethrough. That said, when the septum 136 or 138 is not intact (i.e., when the septum 136 or 138 includes a slit, a plurality of intersecting slits, or a through hole as set forth above), the distal portion of the distal sleeve 204 of the handle 198 can be used to deliver the access guidewire 110 through the septum 136 or 138. Notably, the distal sleeve 204 of the handle 198 can further include one or more ‘O’-rings or the like therein, the one-or-more ‘O’-rings or the like configured to seal the access guidewire 110 in the distal sleeve 204. With the one-or-more ‘O’-rings or the like sealing the access guidewire 110 in the distal sleeve 204, a vacuum can be easily established and maintained with the syringe 106 during the blood-aspirating step set forth below—even when the septum-piercing tip 210 or 212 or the distal portion of the distal sleeve 204 is disposed across the septum 136 or 138.

The gap 206 between the proximal and distal sleeves 202 and 204 of the handle 198 is configured to provide access to the access guidewire 110 in the gap 206 for handling the access guidewire 110. Indeed, by way of the gap 206, the access guidewire 110 can be grasped between a thumb and one or more fingers of a hand and advanced into the introducer adapter 102, needle 104, blood vessel, etc. by pushing it into a proximal opening of the distal sleeve 204. Alternatively, the access guidewire 110 can be pressed into a segmented crescent-shaped thumbpad 214 by the thumb and, with continued pressing, slid across the thumbpad 214 into the proximal opening of the distal sleeve 204 while cradling the handle 198 with the one-or-more fingers of the same hand. Likewise, by way of the gap 206, the access guidewire 110 can be grasped between the thumb and the one-or-more fingers of the hand and withdrawn from the introducer adapter 102, needle 104, blood vessel, etc. by pulling it out of the proximal opening of the distal sleeve 204. Alternatively, the access guidewire 110 can be pressed into the thumbpad 214 by the thumb and, with continued pressing, slid across the thumbpad 214 out from the proximal opening of the distal sleeve 204 while cradling the handle 198 with the one-or-more fingers of the same hand.

The guidewire conduit 200 is configured to provide storage of the access guidewire 110 in a sterile state thereof. Indeed, a tubular wall of the guidewire conduit 200 provides a sterile barrier configured to maintain the sterile state of the access guidewire 110 as long as the access guidewire 110 is disposed therein.

### Methods

Methods include at least a method for securing vascular access. Such a method includes one or more steps selected from an introducer assembly-obtaining step, a septum-piercing or -penetrating step, an introducer assembly-adjusting step, a needle tract-establishing step, a plunger-withdrawing step, a blood-aspirating step, an access guidewire-advancing step, a guidewire-management-device-withdrawing step, a syringe-disconnecting step, an air-bleeding step, and a needle-withdrawing step.

The introducer assembly-obtaining step includes obtaining the introducer assembly 100. As set forth above, the introducer assembly 100 includes at least the syringe 106, the needle 104, and the introducer adapter 102 fluidly coupling the syringe 106 and the needle 104 together. The introducer adapter 102 includes the primary conduit 112, the secondary conduit 114 extending from the side of the primary conduit 112, and the valve 116 including, in some embodiments, the septum 136 or 138 sealing the proximal portion of the primary conduit 112 or the secondary conduit 114.

The septum-piercing step includes piercing the septum 136 or 138 with the integral septum-piercing tip 210 of the distal portion of the distal sleeve 204 of the handle 198 or the septum-piercing tip 212 coupled to the distal portion of the distal sleeve 204 of the handle 198. The septum-penetrating step includes penetrating the septum 136 or 138 with the distal portion of the distal sleeve 204 of the handle 198 when the septum 136 or 138 includes the slit, the plurality of intersecting slits, or the through hole set forth above. The septum-piercing or -penetrating step is performed before the introducer assembly-adjusting step or after the needle-tract establishing step such as after the blood-aspirating step or during the access guidewire-advancing step as set forth below.

Notably, the access guidewire 110 can be used directly without the guidewire-management device 108 when the introducer adapter 102 includes the valve 132. In such embodiments, the method can include an access guidewire-inserting step in place of the septum-piercing or -penetrating step. The access guidewire-inserting step includes inserting the access guidewire 110 into the introducer adapter 102 through the valve 132 when open, which step is followed by closing the valve 132 either immediately after inserting the access guidewire 110 therethrough or at some time before the blood-aspirating step if the blood-aspirating step is to be performed.

The introducer assembly-adjusting step includes adjusting the introducer assembly 100 such that the introducer assembly 100 is in the ready-to-deploy state thereof if not already upon performing the introducer assembly-obtaining step. In the ready-to-deploy state of the introducer assembly 100, the guidewire tip 192 of the access guidewire 110 is just proximal of the needle tip 180 in the distal portion of the needle shaft 176 of the needle 104. This allows for the advancing of the access guidewire 110 into the blood-vessel lumen immediately upon the establishing of the needle tract.

The needle tract-establishing step includes establishing a needle tract from an area of skin to a blood-vessel lumen of a patient with the needle 104.

The plunger-withdrawing step includes withdrawing the plunger 150 from the barrel 148 of the syringe 106 to create a slight vacuum before reaching the blood-vessel lumen in the needle tract-establishing step. The slight vacuum ensures blood flashes back into at least the syringe tip 152 to confirm the establishing of the needle tract in the needle tract-establishing step.

The blood-aspirating step includes aspirating blood with the syringe 106 to confirm the establishing of the needle tract. The seal of the septum 136 or 138 or the like around either the distal portion of the distal sleeve 204 of the handle 198, particularly when the distal sleeve 204 includes the one-or-more ‘O’-rings or the like disposed therein, or the bare-wire portion 194 of the access guidewire 110 is sufficient to maintain a vacuum during the blood-aspirating step.

The access guidewire-advancing step includes advancing at least the guidewire tip 192 of the access guidewire 110 into the blood-vessel lumen for the securing of the vascular access. However, in some embodiments, the septum-piercing or -penetrating step intervenes the access guidewire-advancing step. Indeed, after advancing a few centimeters of the access guidewire 110 or the guidewire tip 192 thereof into the blood-vessel lumen, the septum-piercing or -penetrating step can be performed to eliminate any friction on the access guidewire 110 by the septum 136 or 138 making it easier to perform a remainder of the access guidewire-advancing step. Regardless, the advancing of the access guidewire 110 into the blood-vessel lumen includes grasping the access guidewire 110 between a thumb and one or more fingers of a hand and pushing it into the proximal opening of the distal sleeve 204. Alternatively, the access guidewire 110 can be pressed by the thumb into the thumbpad 214 in the gap 206 of the handle 198 and, with continued pressing, slid across the thumbpad 214 into the proximal opening of the distal sleeve 204 while cradling the handle 198 with the one-or-more fingers of the same hand. Notably, the access guidewire-advancing step allows the guidewire tip 192 to transition from the straightened state in the needle 104 to the curved state in the blood-vessel lumen.

The guidewire-management-device-withdrawing step includes withdrawing the guidewire-management device 108 from the introducer adapter 102 after the access guidewire-advancing step—if the guidewire-management device 108 is used in the method. The guidewire-management-device-withdrawing step leaves the septum 136 or 138 to seal around the bare-wire portion 194 of the access guidewire 110.

The syringe-disconnecting step includes disconnecting the syringe 106 before the needle-withdrawing step, thereby preventing creation of a vacuum during the needle-withdrawing step. That said, the air-bleeding step can obviate the syringe-disconnecting step as the air-bleeding step includes bleeding air into the valve 116 while performing the needle-withdrawing step. Bleeding the air into the valve 116 can include pushing the access guidewire 110 to a side of the valve 128 or 130) to deform the septum 136 or 138; however, in some embodiments, the introducer adapter 102 includes a push-button bleed valve for the air-bleeding step. Notwithstanding the foregoing, both the syringe-disconnecting step and the air-bleeding step can be performed in some embodiments of the method.

The needle-withdrawing step includes withdrawing the needle 104 from the patient but leaving the access guidewire 110 in the blood-vessel lumen. The needle-withdrawing step includes withdrawing both the introducer adapter 102 and the needle 104 over the proximal portion of the access guidewire 110 until a proximal end of the access guidewire 110 escapes from the needle 104. Notably, the needle-withdrawing step includes holding the access guidewire 110 in place at or near the area of skin including the needle tract while withdrawing both the introducer adapter 102 and the needle 104 over the proximal portion of the access guidewire 110.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An introducer assembly, comprising:
a syringe;
a needle;
an introducer adapter fluidly coupling the syringe and the needle together, the introducer adapter including:
a primary conduit;
a secondary conduit extending from a side of the primary conduit; and
a valve including an elastomeric septum sealing a proximal portion of the primary conduit or the secondary conduit; and
an access guidewire optionally loaded in the introducer adapter and sealed therein by the valve.

2. The introducer assembly of claim 1, wherein the valve is a non-elastomeric cylindrical or conical insert with the septum sealing an opening of the insert, the valve disposed in the proximal portion of the primary conduit or the secondary conduit.

3. The introducer assembly of claim 2, wherein the septum is recessed in the proximal portion of the primary conduit or the secondary conduit.

4. The introducer assembly of claim 1, wherein the valve is an elastomeric grommet with the septum integral therewith sealing a cavity of the grommet, the valve disposed in the proximal portion of the primary conduit or the secondary conduit.

5. The introducer assembly of claim 1, wherein the valve seals the proximal portion of the secondary conduit.

6. The introducer assembly of claim 1, wherein the valve seals the proximal portion of the primary conduit.

7. The introducer assembly of claim 1, wherein an introducer-adapter bore in the proximal portion of the primary conduit includes a Luer taper complementing that of a syringe tip of the syringe.

8. The introducer assembly of claim 1, wherein an introducer-adapter bore in the proximal portion of the secondary conduit includes a Luer taper complementing that of a syringe tip of the syringe.

9. The introducer assembly of claim 1, wherein an introducer-adapter tip in a distal portion of the introducer adapter includes a Luer taper complementing that of a needle-hub bore of a needle hub of the needle.

10. The introducer assembly of claim 1, further comprising a guidewire-management device including the access guidewire, the guidewire-management device optionally coupled to the introducer adapter.

11. The introducer assembly of claim 10, the guidewire-management device further including:
a handle including a proximal sleeve, a distal sleeve, and a gap between the proximal and distal sleeves configured to provide access to the access guidewire for handling the access guidewire; and
a guidewire conduit coupled to the proximal sleeve, the guidewire conduit configured to provide storage of the access guidewire in a sterile state thereof.

12. The introducer assembly of claim 11, wherein a distal portion of the distal sleeve includes an integral septum-piercing tip configured to pierce the septum of the valve and deliver the access guidewire therethrough.

13. The introducer assembly of claim 11, the guidewire-management device further including a septum-piercing tip coupled to a distal portion of the distal sleeve, the septum-piercing tip configured to pierce the septum of the valve and deliver the access guidewire therethrough.

14. The introducer assembly of claim 1, wherein the access guidewire includes a bare-wire portion around which the septum seals.

15. The introducer assembly of claim 1, wherein the access guidewire includes a wound-wire portion terminating with an atraumatic ‘J’-shaped guidewire tip.

16. An introducer adapter, comprising:
a primary conduit;
a secondary conduit extending from a side of the primary conduit; and
a valve including an elastomeric member for sealing a proximal portion of the primary conduit or the secondary conduit.

17. The introducer adapter of claim 16, wherein the valve is a non-elastomeric cylindrical or conical insert with an elastomeric septum for sealing an opening of the insert, the valve disposed in the proximal portion of the primary conduit or the secondary conduit.

18. The introducer adapter of claim 17, wherein the septum is recessed in the proximal portion of the primary conduit or the secondary conduit.

19. The introducer adapter of claim 16, wherein the valve is an elastomeric grommet with an elastomeric septum integral therewith for sealing a cavity of the grommet, the valve disposed in the proximal portion of the primary conduit or the secondary conduit.

20. The introducer adapter of claim 16, wherein the valve is an integrated Touhy-Borst valve including an elastomeric ‘O’-ring for sealing the valve, the valve integrated into the proximal portion of the primary conduit or the secondary conduit.

21. The introducer adapter of claim 16, wherein the valve is a Touhy-Borst adapter including an elastomeric ‘O’-ring for sealing the valve, the adapter coupled to the proximal portion of the primary conduit or the secondary conduit.

22. The introducer adapter of claim 16, wherein the valve seals the proximal portion of the secondary conduit.

23. The introducer adapter of claim 16, wherein the valve seals the proximal portion of the primary conduit.

24. The introducer adapter of claim 16, wherein an introducer-adapter bore in the proximal portion of the primary conduit includes a Luer taper complementing that of a syringe tip of the syringe.

25. The introducer adapter of claim 16, wherein an introducer-adapter bore in the proximal portion of the secondary conduit includes a Luer taper complementing that of a syringe tip of the syringe.

26. The introducer adapter of claim 16, wherein an introducer-adapter tip in a distal portion of the introducer adapter includes a Luer taper complementing that of a needle-hub bore of a needle hub of the needle.

27. A method for securing vascular access, comprising:
obtaining an introducer assembly, the introducer assembly including:
a syringe;
a needle;
an introducer adapter fluidly coupling the syringe and the needle together, the introducer adapter including:
a primary conduit;
a secondary conduit extending from a side of the primary conduit; and
a valve including an elastomeric septum sealing a proximal portion of the primary conduit or the secondary conduit;
establishing a needle tract from an area of skin to a blood-vessel lumen of a patient with the needle; and
advancing at least a guidewire tip of an access guidewire into the blood-vessel lumen for the securing of the vascular access.

28. The method of claim 27, further comprising adjusting the introducer assembly such that the introducer assembly is in a ready-to-deploy state thereof with the guidewire tip of the access guidewire just proximal of a needle tip in a distal portion of a needle shaft of the needle for the advancing of the access guidewire into the blood-vessel lumen immediately upon the establishing of the needle tract.

29. The method of claim 28, further comprising piercing the septum with an integral septum-piercing tip of a distal portion of a distal sleeve of a guidewire-management device before the adjusting of the introducer assembly.

30. The method of claim 29, wherein the advancing of the access guidewire into the blood-vessel lumen allows the guidewire tip to transition from a straightened state in the needle to a curved state in the blood-vessel lumen.

31. The method of claim 29, further comprising withdrawing the guidewire-management device from the introducer adapter after the advancing of the access guidewire into the blood-vessel lumen, the withdrawing of the guidewire-management device from the introducer adapter leaving the septum to seal around a bare-wire portion of the access guidewire.

32. The method of claim 31, further comprising aspirating blood with the syringe to confirm the establishing of the needle tract, the seal of the septum around the bare-wire portion of the access guidewire sufficient to maintain a vacuum during the aspirating of the blood with the syringe.

33. The method of claim 27, further comprising withdrawing the needle from the patient but leaving the access guidewire in the blood-vessel lumen, the withdrawing of the needle from the patient including withdrawing both the introducer adapter and the needle over a proximal portion of the access guidewire until a proximal end of the access guidewire escapes from the needle.

34. The method of claim 33, wherein the withdrawing of the needle from the patient includes holding the access guidewire in place at or near the area of skin including the needle tract while withdrawing both the introducer adapter and the needle over the proximal portion of the access guidewire.

35. The method of claim 33, further comprising disconnecting the syringe before the withdrawing of the needle from the patient, thereby preventing creation of a vacuum during the withdrawing of the needle from the patient.
